(19) United States
(12) Patent Application Publication
(10) Pub. No.: US 2012/0221092 A1
(43) Pub. Date: Aug. 30, 2012

(54) CATHETER GRIPPER

(75) Inventors: Ronen Jaffe, Doar-Na Misgav (IL); Eytan Jaffe, Pardes Chana-Karkur (IL); Eliahu Eliachar, Haifa (IL); Nir Lilach, Kfar Yehoshua (IL)

(73) Assignee: CARDIOFLOW LTD., M.P. Misgav (IL)

(21) Appl. No.: 13/508,360

(22) PCT Filed: Nov. 5, 2010
(86) PCT No.: PCT/IB10/55022

§ 371 Date: May 5, 2012

Related U.S. Application Data
(60) Provisional application No. 61/258,241, filed on Nov. 5, 2009.

(51) Int. Cl. A61F 2/84; A61M 25/10; A61M 25/16

(52) U.S. Cl. 623/1.11; 604/533; 604/96.01

(57) ABSTRACT

A fastener for attaching a device to a catheter used to mediate a procedure in a patient's body, the fastener dimensioned so that it and the device can be introduced into the patient's body with the catheter and comprising: a clutch controllable to lock the fastener onto and to unlock the fastener from the catheter; and a controller operable to control the clutch selectively to Clock onto and unlock from the catheter when the catheter and fastener are inside a body of a patient.

### CROSS-REFERENCES TO RELATED APPLICATIONS

The present application claims benefit under 35 U.S.C. §119(e) of US Provisional Application 61/258,241 filed Nov. 5, 2009 the entire content of which is incorporated herein by reference.

### TECHNICAL FIELD

Embodiments of the invention relate to methods and apparatus for attaching devices to a catheter so that the devices may be introduced into a body lumen together with the catheter.

### BACKGROUND

Minimally invasive procedures for treating a bodily vessel typically involve delivering interventional, diagnostic and/or therapeutical devices to a treatment site in the vessel using tubes, referred to as catheters, that are configured for insertion into the body though a natural or artificial orifice formed in the body. Blood vessels, regions of the gastrointestinal tract, and the urethra, are examples of vessels that are treated using minimally invasive procedures.

Among well-known minimally invasive procedures mediated with catheters are procedures for deploying stents to alleviate coronary blood vessel blockages, referred to as “stenoses”, resulting from thromboses (blood clots) or the buildup of plaque that restrict or completely block blood flow. A stent is a tube whose walls are generally formed from a metal mesh, which is introduced into the body in a collapsed state characterized by a small cross section diameter and positioned in a stenosis blocking blood flow in a blood vessel to be treated. When properly positioned the stent is expanded to increase its diameter so that it presses open the stenosis and enables enhanced blood flow through the vessel.

A stent deployment procedure to alleviate blockage of blood flow through an artery compromised by a stenosis typically comprises introducing a guiding catheter into the body through an (artificial) opening formed in the femoral artery and threading the catheter through the vascular system until a distal end of the catheter is located near the stenosis. A steerable guide wire is introduced through the guiding catheter and steered to and though the region of the stenosis. A stent deployment device comprising its own “deployment catheter” having a stent mounted at its distal end is threaded along the guide wire until the stent is positioned in the stenosis. Opening the positioned stent opens the stenosis and returns blood flow though the artery.

Procedures comprising deploying a stent in an artery often involve additional procedures such as imaging a stenosis in a treated artery before, and/or after stenting, and capturing and removing debris, such as small granular calcium deposits or embolisms, that are produced and released into the blood during a stent deployment process.

Imaging before stent deployment in an artery, or other blood vessel may be performed to visualize a condition of a stenosis, a region of the blood vessel in which it is located and/or to better understand the location of the stenosis in the blood vessel. Imaging after deployment may be performed to determine if the stent is properly positioned and expanded. If not properly deployed, corrective procedures may be indicated. Imaging is often done using an ultrasound imager referred to as an “intravascular ultrasound imaging system (IVUS)” introduced into the artery.

If not captured and removed, debris introduced into the blood stream during deployment of a stent to treat a stenosis, can cause dangerous blood flow blockages in blood vessels downstream of the stenosis. An embolism protection device (EPD) introduced into the treated artery is used to capture and remove potentially dangerous debris. Some EPDs, referred to as “proximal EPDs”, comprise an occlusion balloon and aspirator that are introduced by a catheter into the artery proximal to the stenosis. Inflating the occlusion balloon, temporarily to block blood flow in the artery during stent deployment, and controlling the aspirator to aspirate the blood, removes debris generated by the deployment. In some EPDs, referred to as “distal EPDs”, a filter is positioned distal to the stenosis to trap debris that flows downstream of the stenosis with the blood. Upon completion of the deployment procedure, the filter and the debris with it, are removed from the artery.

The various devices used in stenting and other catheter mediated procedures are generally comprised in an integrated system that includes components, such as a guide catheter and guide wire for introducing and manipulating the devices inside the body, which are matched to the devices and their applications. Catheter procedures, such as stenting, are complicated and delicate procedures that require practiced skill and intimate, dexterous familiarity with the catheters and tools used to perform the procedures acquired from repeated performances of the procedures and use of the tools. Surgeons and other professionals that work together in performing the procedures therefore typically develop strong preferences for using tools with which they have become familiar through repeated use, and biases against using tools with which they have less experience. As a result, surgeons and professionals tend to omit potentially advantageous functions if they are mediated by catheters, guide wires, and associated tools other than the ones with which they have intimate experience.

Examples of catheter procedures and devices used in the procedures are described in the following documents.

WO 2009/050599 “GUIDEWIRE STOP” relates to “an actuatable guidewire stop configured to limit movement of an intravascular device relative to a guidewire, comprising: a locking tube disposed about the guidewire and having a locked configuration, wherein the locking tube is prevented from movement relative to the guidewire, and an unlocked configuration, wherein the locking tube is moveable relative to the guidewire; a locking element disposed between the guidewire and the locking tube frictionally engaging with at least the guidewire in the locked configuration; and an actuator operatively coupled to the locking element for changing the locking tube from the unlocked configuration to the locked configuration.”

WO 2009/050600 “GUIDEWIRE STOP” relates to “an actuatable guidewire stop configured to limit movement of an intravascular device relative to a guidewire, comprising: a coil spring having an inner lumen with a first diameter configured to slidably and rotationally receive the guidewire in a locked configuration, wherein in the locked configuration the inner lumen has a second diameter smaller than a first diameter.”

### SUMMARY

An embodiment of the invention provides a fastener, hereinafter referred to as a “gripper”, for attaching a device to a catheter that can be mounted to the catheter and inserted into a body with the catheter, and when inside the body be controlled to selectively grip or release the catheter. When controlled to grip the catheter, the gripper is locked to the catheter at a given location along the length of the catheter. When controlled to release the catheter, the gripper is unlocked from the catheter and translatable along the length of the catheter to be controlled to grip and lock itself, and the device, at a new location along the catheter. Control lines that extend along the catheter enable control of the gripper and a device it “carries”, from a proximal end of the catheter when the catheter and gripper are located inside a body.

In an embodiment of the invention, the gripper, is configured so that it can be clipped on to the catheter. Optionally, the gripper is configured so that it is mountable to the catheter by threading the catheter through the gripper.

In an embodiment, the gripper comprises a gripping clutch controllable to selectively grip and lock the gripper to the catheter or release and unlock the gripper from the catheter. Optionally, the gripping clutch comprises a resilient element having first and second states. At least one of the states is a state in which the elastic element exhibits strain. Optionally, one of the states is a state in which the elastic element is relaxed and does not exhibit strain.

In one of the states of the resilient element, the clutch, and thereby the gripper, grip the catheter and in the other state, the gripper does not grip the catheter. Optionally, in the state for which the gripper grips the catheter, the resilient element presses on the catheter to generate frictional forces that prevent motion of the gripper along the catheter. Optionally, the resilient element comprises a spring. In some embodiments of the invention, the gripping clutch comprises an inflatable element, which when inflated generates frictional forces that lock the gripper to the catheter and when deflated reduces the frictional forces to unlock the gripper.

A method of using a gripper, in accordance with an embodiment of the invention, comprises: attaching the gripper comprising a device for performing a procedure in a body to a distal end of a catheter; introducing the distal end into a body; and using the control line to adjust the position of the gripper and device on the catheter. Examples of devices that may be carried into a body by a gripper in accordance with an embodiment of the invention are occlusion balloons, aspirators, ultrasound and/or optical imaging devices, ostial positioners, and tissue ablators.

There is therefore provided in accordance with an embodiment of the invention a fastener for attaching a device to a catheter used to mediate a procedure in a patient's body, the fastener dimensioned so that it and the device can be introduced into the patient's body with the catheter and comprising: a clutch controllable to lock the fastener onto and to unlock the fastener from the catheter; and a controller operable to control the clutch selectively to lock onto and unlock from the catheter when the catheter and fastener are inside a body of a patient. Optionally, the controller is operable to reposition the clutch from a first location along the catheter when the clutch is unlocked and locate and lock the clutch and fastener at a new second location along the catheter. Additionally or alternatively, the fastener is optionally formed having a slot through which the catheter may be pressed to seat the catheter in the clutch.

In an embodiment of the invention, the clutch comprises an inflatable element, which when inflated generates frictional forces that lock the fastener to the catheter and when deflated reduces the frictional forces to unlock the fastener from the catheter. Optionally, the controller comprises an inflation tube that is connected to the inflatable element through which fluid is pumped into or released out from the inflatable element from outside the body to respectively lock or unlock the fastener. Optionally, the inflation tube has sufficient stiffness so when the clutch is unlocked, the tube is useable to pull the fastener in a proximal direction or push the fastener in a distal direction along the catheter.

In an embodiment of the invention, the clutch comprises a resilient element having first and second states in one of which states the clutch is locked onto the catheter, and in the other of the states the clutch is unlocked from the catheter. Optionally, the resilient element comprises a compressive element, which in a first state generates frictional forces that lock the fastener to the catheter and in the second state unlocks the fastener from the catheter. Additionally or alternatively, the resilient element comprises a wire form having surface regions that press on the catheter in the first state to generate the frictional forces. Optionally, in the second state, the compressive element is compressed relative to the first state. Optionally, the relative compression that characterizes the second state deforms the resilient element to increase distances between the surface elements of the spring to reduce the frictional forces.

In an embodiment of the invention, the wire form comprises two pairs of wire arms joined at a junction to form an X. Optionally, the surface regions are surface regions of the arms of each pair that press on opposite sides of the catheter to generate the frictional forces. Optionally, compressing the wire form moves the surface regions of the arms in a pair of arms away from each other.

In an embodiment of the invention, the wire form comprises opposite facing U-shaped loops curved to press on the catheter from opposite sides of the catheter to generate the frictional forces. Optionally, when compressed relative to the first state, a radius of curvature of the loops increase to reduce the frictional forces. In an embodiment of the invention, the fastener comprises a cowling that houses the clutch. Optionally, the fastener comprises a cowling mount on which the cowling is mounted so that it can slide back and forth along the cowling mount. Optionally, the resilient element is supported between the cowling and cowling mount. Optionally, the controller comprises a control cable housed in a sheath. Optionally, the sheath and control cable are connected to the cowling mount and cowling respectively, and tensile force applied to the cable that translates the control cable relative to the sheath in a direction away from the cowling unlocks the clutch.

In an embodiment of the invention, the fastener has an outer diameter less than or equal to about 8 mm Optionally, the fastener has an outer diameter less than or equal to about 6 mm Optionally, the fastener has an outer diameter less than or equal to about 4 mm Optionally, the fastener has an outer diameter less than or equal to about 2 mm Optionally, the fastener has an outer diameter equal to about 1.68 mm.

In an embodiment of the invention, the fastener comprises an occlusion balloon and aspiration tube. In an embodiment of the invention, the fastener comprises an ostial positioning device. In an embodiment of the invention, the fastener comprises an ultrasound transducer configured to image internal regions of the body.

There is further provided, in accordance with an embodiment of the invention a fastener for attaching a device to a catheter used to mediate a procedure in a patient's body, the fastener dimensioned so that the fastener and device can be introduced into the patient's body with the catheter and comprising: a collar attachable to the catheter so that it freely translatable along the catheter; a clutch attachable to the catheter and controllable to selectively lock onto and be unlocked from the catheter; and a connector that connects the collar and the clutch and maintains a substantially constant distance between them when both are attached to the catheter and the catheter is located in a guide catheter; wherein the connector is sufficiently long so that the collar and clutch may be simultaneously located neat distal and proximal ends of the catheter respectively.

There is further provided, in accordance with an embodiment of the invention a method of deploying a stent to treat a stenosis in a blood vessel, the method comprising: introducing a deployment catheter comprising a stent, an occlusion balloon and an aspiration tube into the blood vessel; while in the blood vessel adjusting a position of the balloon along the catheter relative to a position of the stent; positioning the stent in the stenosis; inflating the occlusion balloon to block flow through the blood vessel; deploying the stent; and aspirating blood in the blood vessel.

There is further provided, in accordance with an embodiment of the invention a method of deploying a stent to treat a stenosis in a blood vessel, the method comprising: introducing a deployment catheter comprising a stent, an aspiration tube, and an occlusion balloon locked to the catheter and located adjacent to the stent into the blood vessel; inflating the occlusion balloon to block flow through the blood vessel and lock the balloon and catheter to the blood vessel; while inflated, unlocking the occlusion balloon from the catheter; moving the catheter to position the stent in the stenosis; deploying the stent; and aspirating blood in the blood vessel.

This Summary is provided to introduce a selection of concepts in a simplified form that are further described below in the Detailed Description. This Summary is not intended to identify key features or essential features of the claimed subject matter, nor is it intended to be used to limit the scope of the claimed subject matter.

### BRIEF DESCRIPTION OF FIGURES

Non-limiting examples of embodiments of the invention are described below with reference to figures attached hereto that are listed following this paragraph. Identical structures, elements or parts that appear in more than one figure are generally labeled with a same numeral in all the figures in which they appear. Dimensions of components and features shown in the figures are chosen for convenience and clarity of presentation and are not necessarily shown to scale.

FIG. 1A schematically shows a stent deployment catheter comprising a stent in accordance with conventional art;

FIG. 1B schematically shows a gripper, in accordance with an embodiment of the invention;

FIG. 1C schematically shows the gripper in FIG. 1B mounted to the deployment catheter shown in FIG. 1A, in accordance with an embodiment of the invention;

FIGS. 1D and 1E schematically show topside and underside views of the gripper shown in

FIG. 1C mounted to and gripping the deployment catheter to lock the gripper to the catheter, in accordance with an embodiment of the invention;

FIGS. 1F and 1G schematically show topside and underside views of the gripper shown in FIG. 1C mounted to, and unlocked from, the deployment catheter so that it is movable along the catheter, in accordance with an embodiment of the invention;

FIGS. 2A-2D schematically show variations of grippers, in accordance with embodiments of the invention;

FIGS. 3A and 3B schematically show a gripper comprising an occlusion balloon and aspirator tube mounted to a stent deployment catheter in accordance with an embodiment of the invention;

FIG. 4A and 4B schematically show a gripper comprising ultrasound imagers for imaging internal features of the body, in accordance with an embodiment of the invention;

FIGS. 5A-5I schematically illustrate using a gripper in a procedure for deploying a stent in accordance with an embodiment of the invention;

FIGS. 6A-6C schematically illustrate using a gripper in another procedure for deploying a stent in accordance with an embodiment of the invention;

FIGS. 7A-7D schematically illustrate a procedure using a gripper comprising an ostial positioning device for deploying a stent in an ostium of a blood vessel in accordance with an embodiment of the invention; and

FIG. 8 schematically shows another gripper, referred to as a split griper, in accordance with an embodiment of the invention.

### DETAILED DESCRIPTION

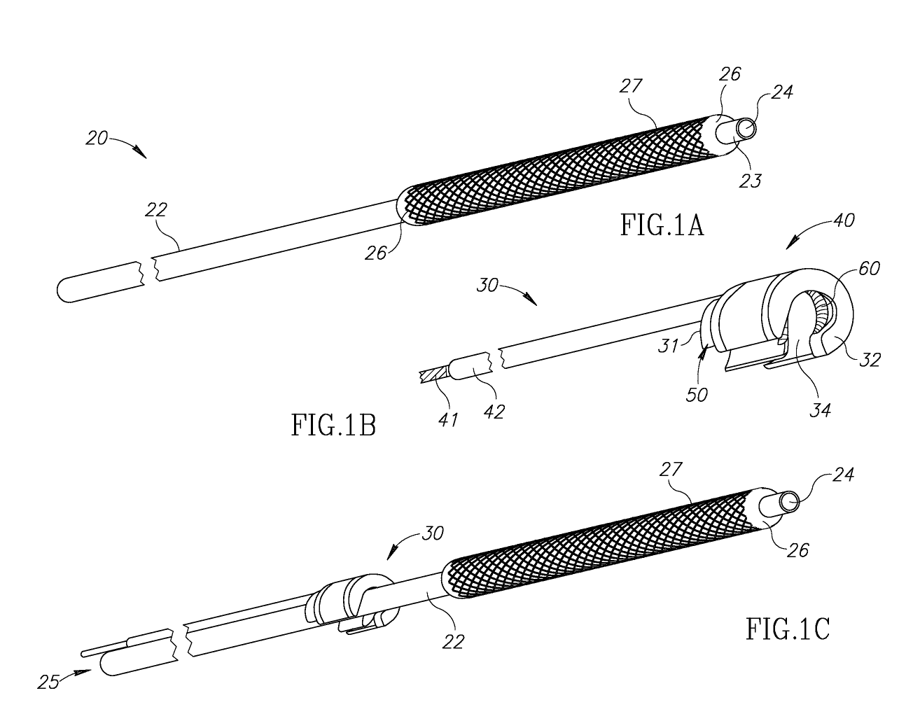

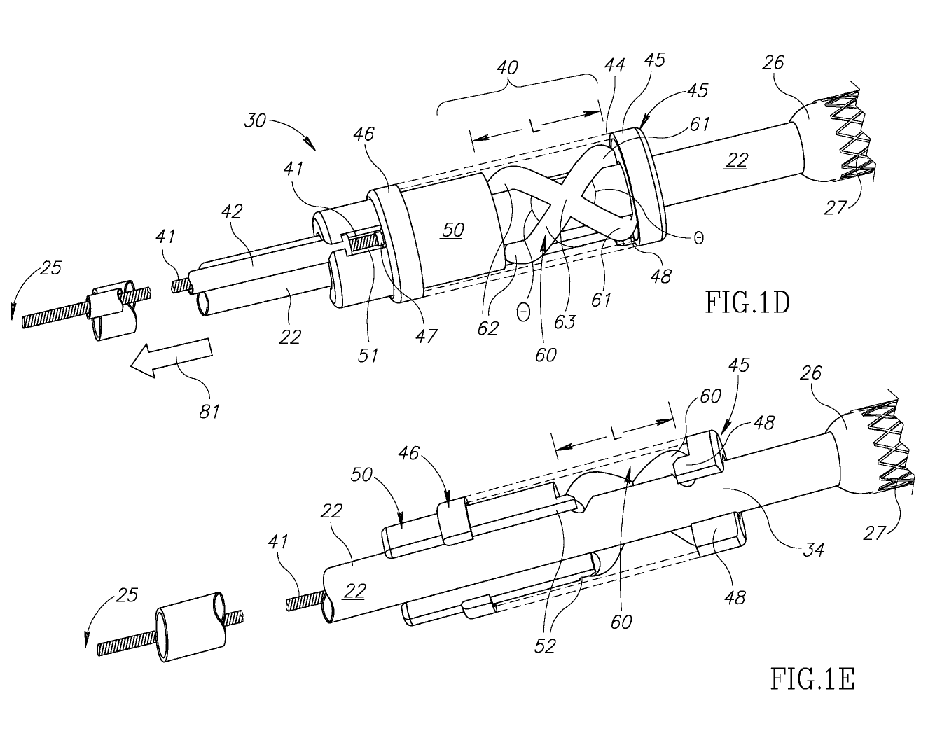

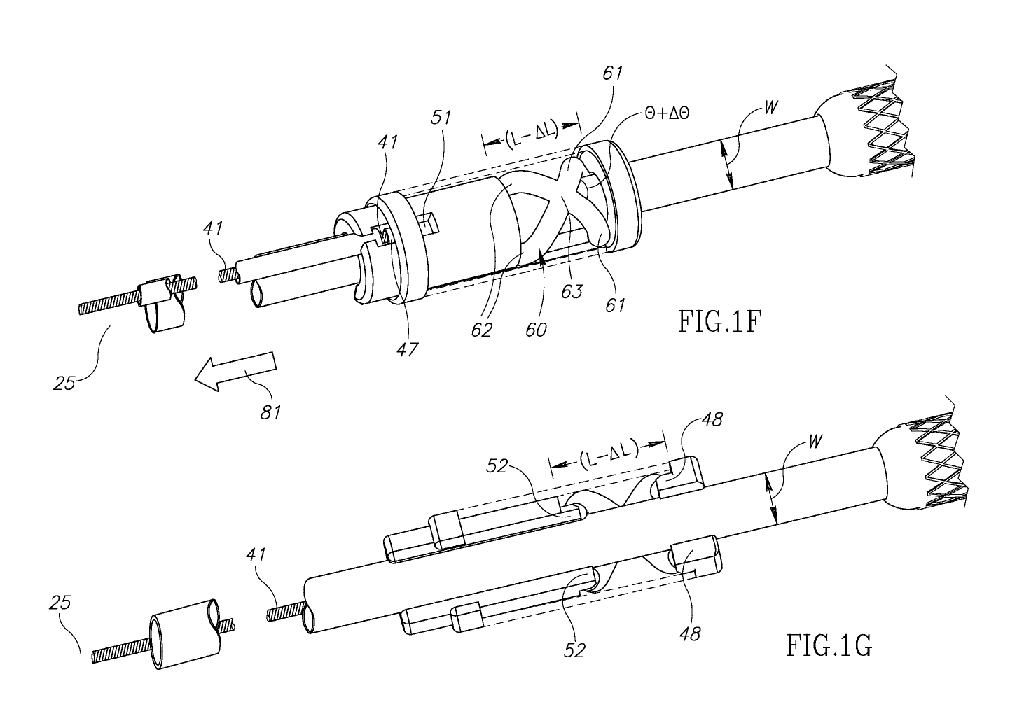

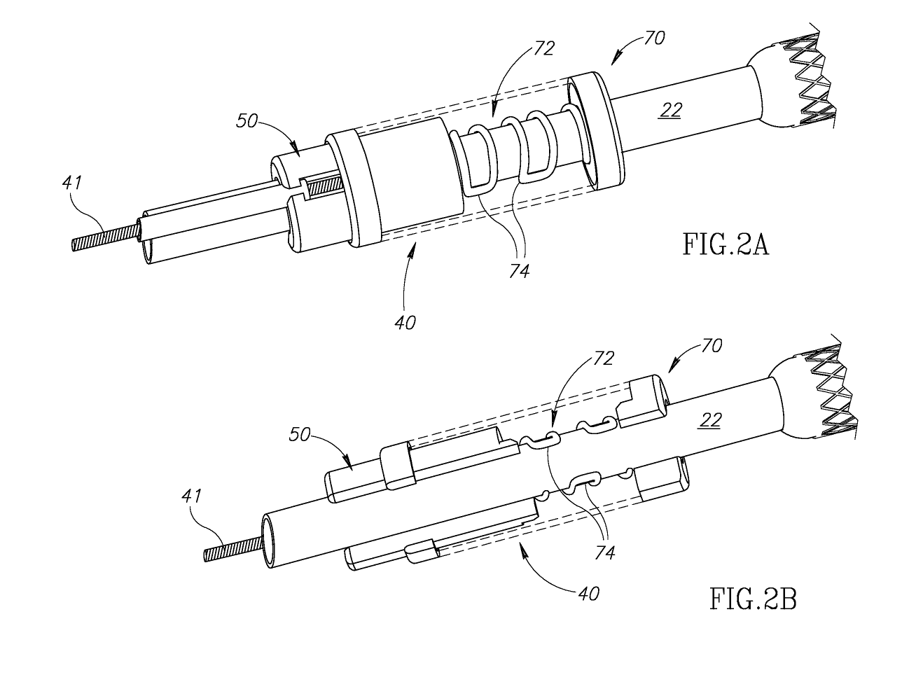

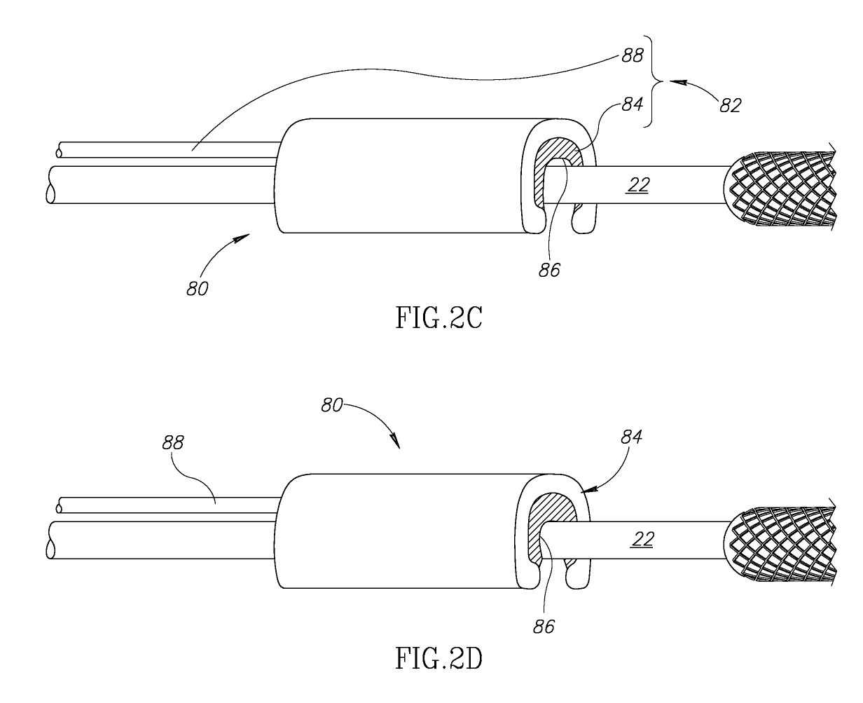

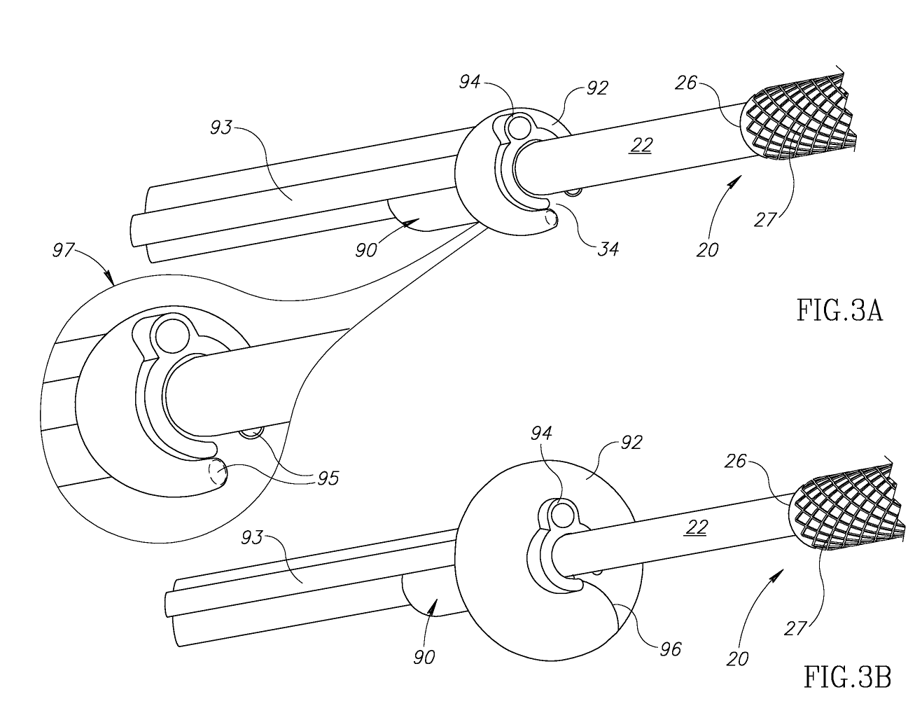

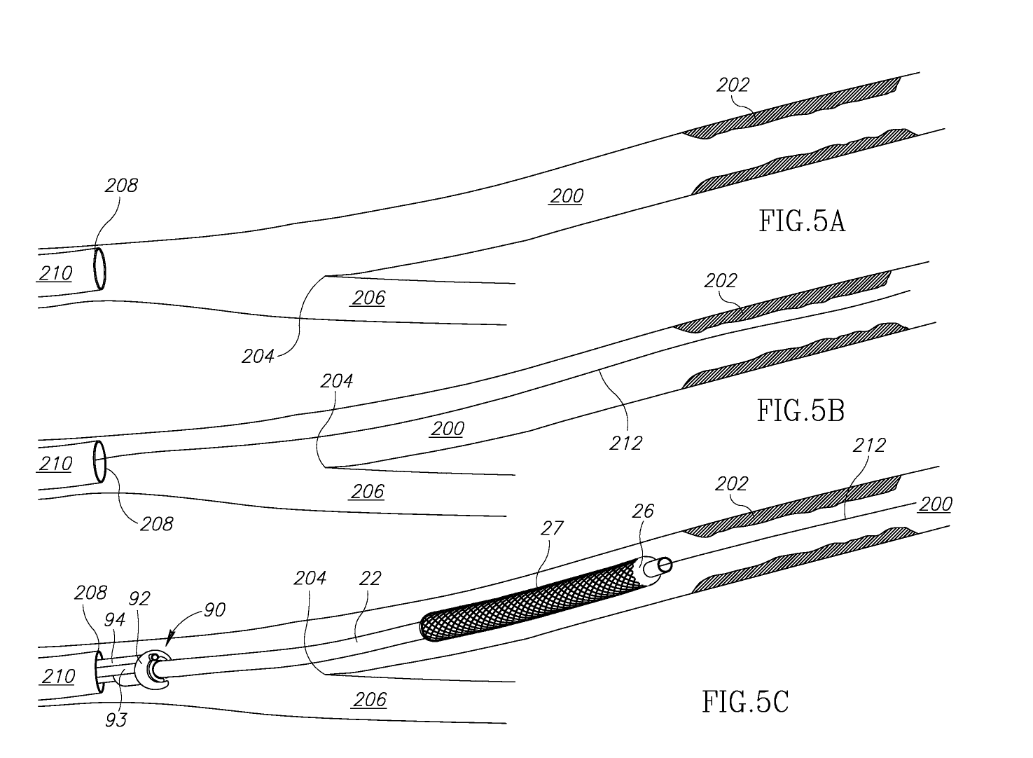

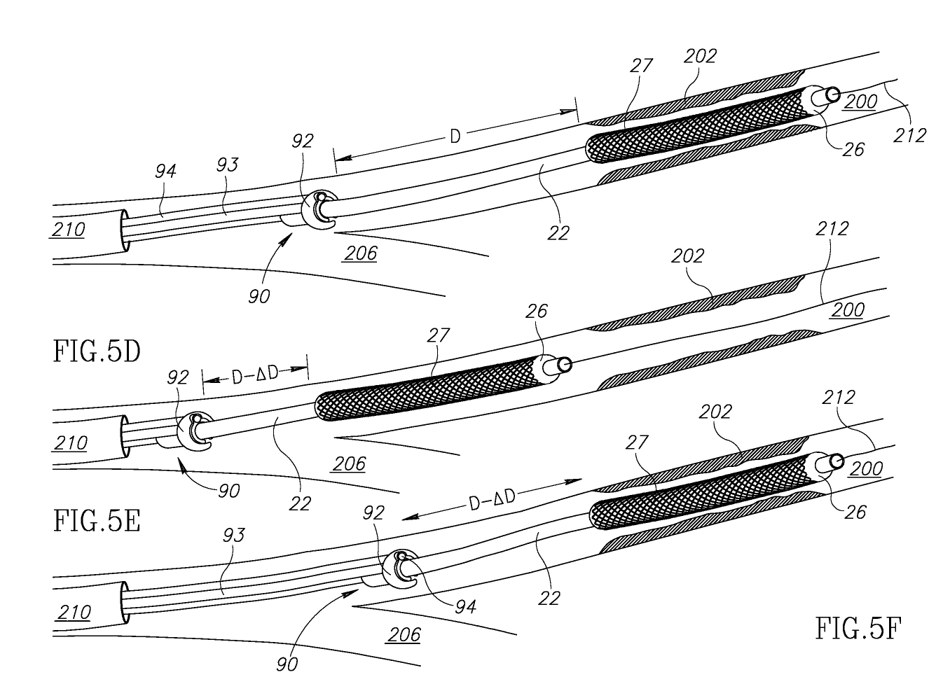

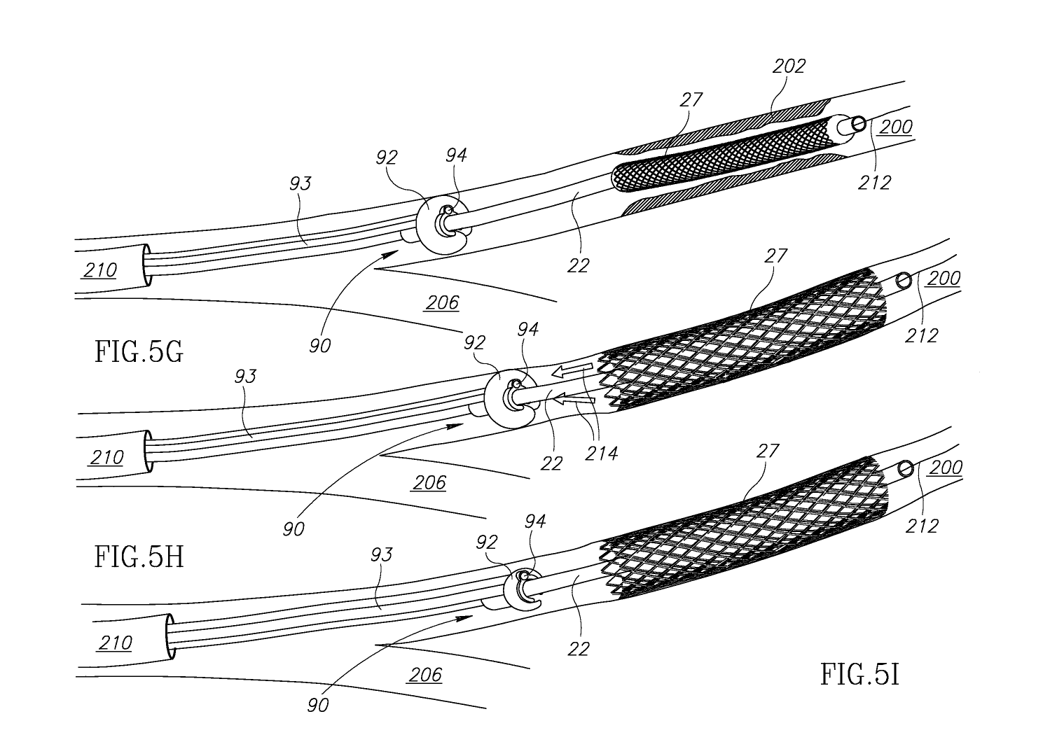

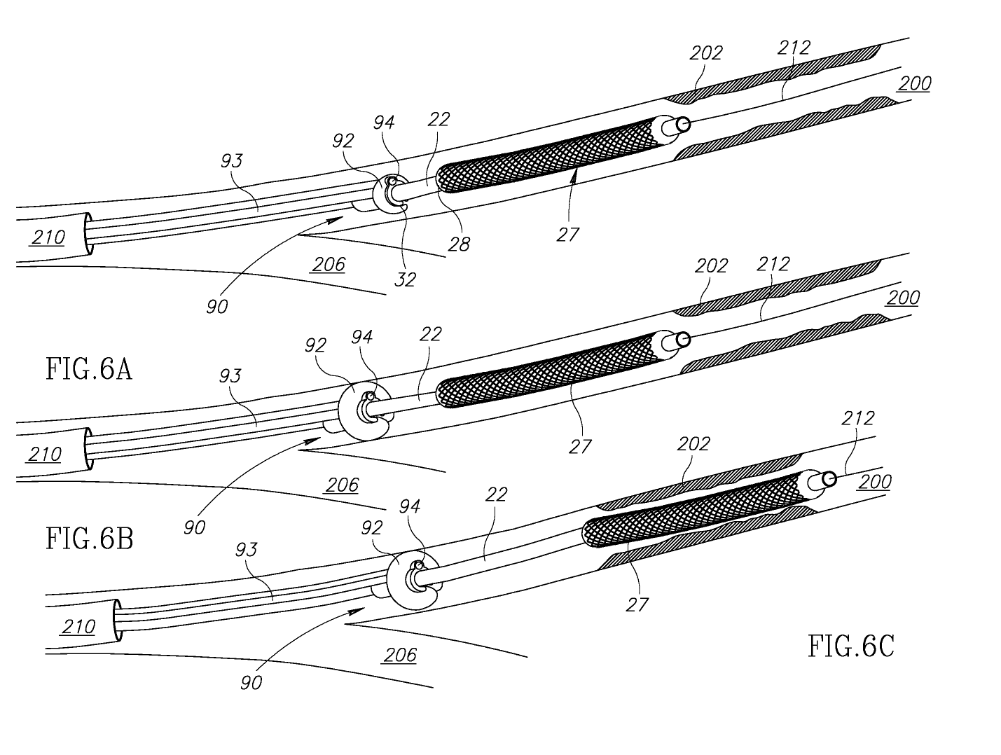

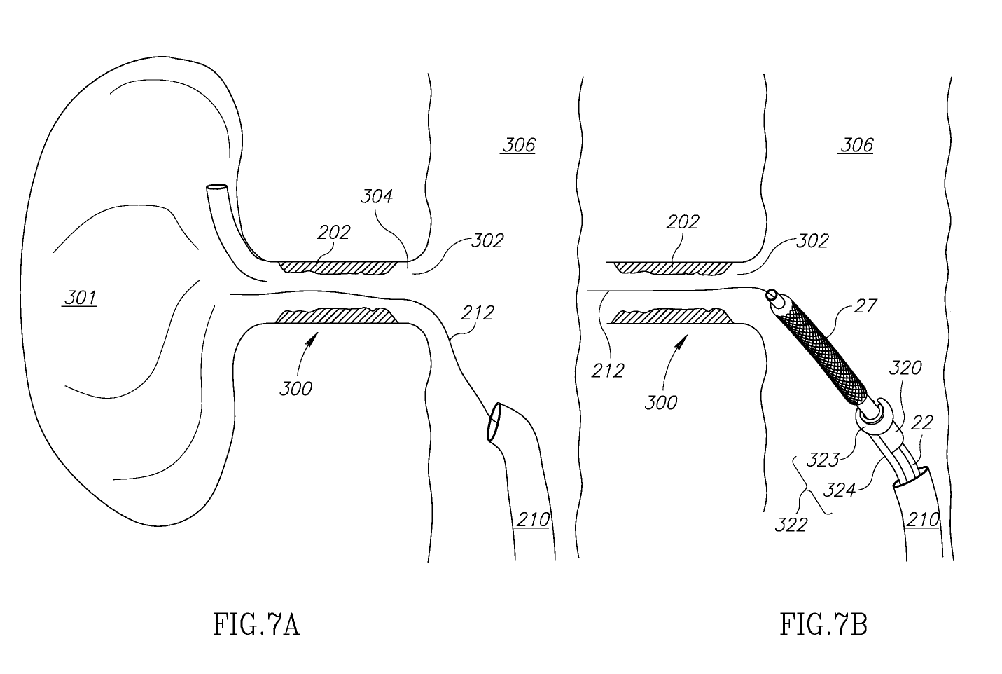

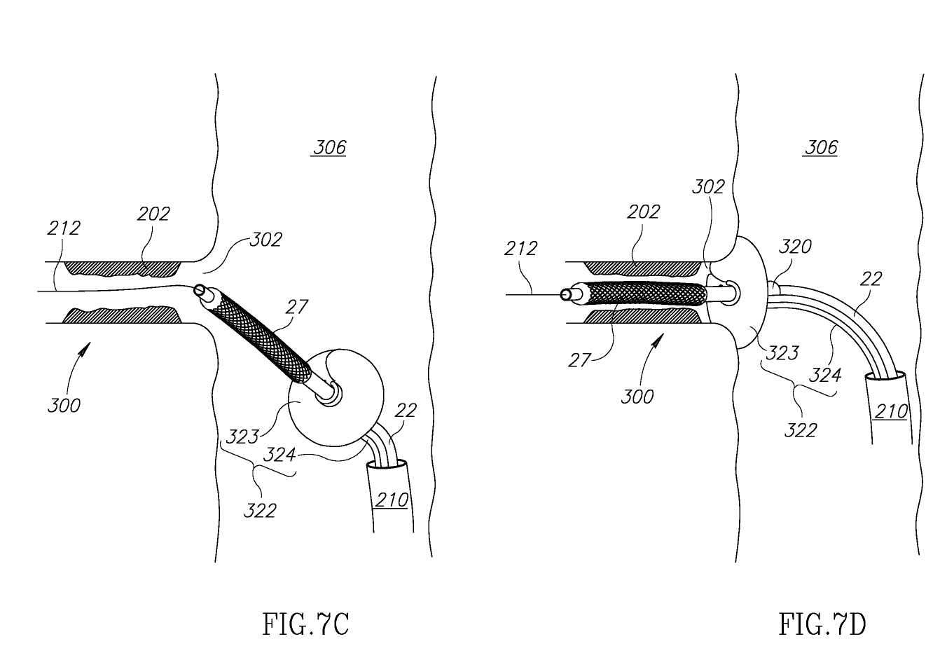

In the detailed description below, aspects of embodiments of the invention are discussed with respect to a stent deployment catheter schematically shown in FIG. 1A and a gripper schematically shown in FIGS. 1B-1G that is attachable to the catheter, in accordance with an embodiment of the invention. Variations of grippers in accordance with embodiments of the invention are discussed with reference to FIGS. 2A-2D. FIGS. 3A, 3B, 4A and 4B, 7A to 7D and 8 schematically shown devices described in the text, which are attachable to grippers in accordance with embodiments of the invention for insertion into a body with a catheter. FIGS. 5A-5I schematically illustrate an example of using a gripper in a procedure to deploy a stent in accordance with an embodiment of the invention. FIGS. 6A-6C schematically illustrate another example of a procedure using a gripper to deploy a stent in accordance with an embodiment of the invention. FIGS. 7A-7D schematically illustrate a procedure using a gripper to deploy a stent to treat an ostial stenosis of a blood vessel in accordance with an embodiment of the invention.

FIG. 1A schematically shows a stent deployment system 20 comprising a stent delivery catheter 22 having an expansion balloon 26 overlaid by a collapsed stent 27 mounted adjacent a distal end 23 of the catheter. Catheter 20 is made from an elastic, biocompatible material suitable for being pushed through the vascular system of a patient to position stent 27 in a stenosis compromising a blood vessel in the patient that is to be treated and opened by deployment of the stent. Stent delivery catheter 22 has a lumen 24, which allows the stent to be threaded over a guide wire, and a balloon inflation lumen (not shown) through which a gas or a liquid may be pumped to inflate balloon 26. Stent delivery catheter is sufficiently long so that when stent 27 is located at a site of a stenosis inside a patient's body, a proximal end 25 of the catheter is located outside of the patient's body.

FIG. 1B schematically shows a gripper 30 configured to be clipped onto a catheter, such as stent delivery catheter 22, in accordance with an embodiment of the invention. Gripper 30 optionally comprises an outer cowling 40 coupled to a cowling mount 50 so that outer cowling 40 is movable, in an axial lengthwise direction, back and forth along the mount. A control cable 41 housed in a sheath 42 extending from a back end of gripper 30 controls relative motion of cowling 40 along mount 50. A back end 31 of gripper 30 from which sheath 42 and control cable 41 extend is referred to as a “proximal” end of the gripper. A front end 32 of gripper 30 is referred to as a “distal” end of the gripper. Optionally, cowling 40 and cowling mount 50 are cylindrical. Control cable 41 and its sheath 42 are long enough so that a proximal end of cable 41 is accessible outside of a patient's body when gripper 30 is located inside the body at a site at which a procedure for which the gripper is introduced into the body is to be performed.

Cowling 40 and mount 50 house a clutch 60, a portion of which is shown shaded in FIG.

1B. Motion of cowling 40 towards proximal end 31 or distal end 32 of gripper 30 controls clutch 60 to respectively release or grip a catheter onto which the gripper is mounted.

Cowling 40, cowling mount 50 and clutch 60 are formed having suitable lengthwise apertures so that gripper 30 is configured having a lengthwise slot 34 through which a catheter is pressed to clip the gripper onto the catheter. In an embodiment of the invention, slot 34, and/or apertures formed in cowling 40, cowling mount 50, and/or clutch 60 are dimensioned so that gripper 30 generates an audible click upon being properly clipped to a catheter. Optionally, the cowling, cowling mount, and/or clutch are configured so that a person clipping the gripper onto a catheter feels a small “jolt” as, and if, the gripper clips properly into place on the catheter. FIG. 1C, by way of example, schematically shows gripper 30 mounted to stent delivery catheter 22. Details of construction and operation of gripper 30 are discussed below with reference to FIGS. 1D-1G.

FIGS. 1D and 1E schematically show enlarged, topside and underside perspective views respectively of gripper 30 clipped onto and gripping catheter 22 so that the gripper is locked onto and not free to move relative to the catheter, in accordance with an embodiment of the invention. The figures show details of cowling 40, cowling mount 50, and clutch 60.

Cowling 40 optionally comprises a cylindrical shell 44, shown in dashed lines, sandwiched between distal and proximal collars 45 and 46 respectively. Shell 44 is shown in dashed lines to indicate that in the figures it is transparent to reveal components and features of gripper 30 underlying the shell, which would normally not be seen in the perspective of the figures. Proximal collar 46 comprises an anchor nub 47 to which control cable 41 is attached. Cowling mount 50 is formed having a slot 51 which receives anchor nub 47 and in which the anchor nub is free to move back and forth. Anchor nub 47 and slot 51 cooperate to maintain cowling 40 and cowling mount 50 aligned and limit range of motion of the cowling relative to the mount to prevent the cowling from sliding off the mount. Cable sheath 42 is fixed to cowling mount 50.

Cowling mount 50 has two support buttons 52, which are clearly shown in the underside perspective view of FIG. 1E. Distal collar 45 of cowling 40 comprises two support buttons 48, one of which is shown in each of FIGS. 1D and 1E, that are, optionally, mirror images of support buttons 52 comprised in cowling mount 50. Clutch 60 is an elastic, optionally X-shaped, elastic element having curved distal arms 61 and curved proximal arms 62 that meet at a junction 63. The distal and proximal arms are supported by support buttons 48 and 52 respectively. X-shaped clutch 60 exerts an elastic force on cowling 40 and cowling mount 50 that operates to push the cowling and mount apart to a maximum displacement allowed by a range of motion of anchor nub 47 in slot 51. If no tensile force is applied to “pull” control cable 41 in a direction indicated by a block arrow 81 and oppose the elastic force of clutch 60, the clutch pushes the cowling and the mount apart to a maximum allowed distance, “L”, and the curved arms of clutch 60 hug deployment catheter 22 tightly, and gripper 30 is locked to the catheter. In the locked state, distal arms 61 make an angle 0, hereinafter referred to as an “inter-arm” angle, with each other at junction 63. Similarly, proximal arms 62 make the same inter-arm angle 0 with each other in the locked state.

Applying a sufficient pulling force in a direction indicated by block arrow 81 on control cable 41, while maintaining sheath 42 fixed relative to the catheter pulls cowling 40 proximally in the direction indicated by the block arrow relative to mount 50 and compresses clutch 60. Under compression, the angle between distal arms 61 and between proximal arms 62 of the clutch increases, the arms respectively move apart and loosen their grip on catheter 22 and unlock gripper 30 from catheter 22. FIGS. 1F and 1G schematically show topside and underside perspective views of gripper 30 unlocked from catheter 22 by a tensile force F applied to control cable 41 in the direction indicated by block arrow 81. FIG. 1F shows anchor nub 47 displaced proximally in slot 51, clutch 60 compressed to a length (L-ΔL), and the angle between distal arms 61 and between proximal arms 62 increased to an angle (θ+Δθ) by force F. For the change in length −ΔL by which clutch 62 is compressed, a distance AD by which distal arms 61 move apart from each other and by which proximal arms 62 move apart from each other to loosen their grip on deployment catheter 22 may be estimated by an expression ΔD=ΔL/ tan (θ/2).

In the unlocked state, gripper 30 can be moved along the catheter to change location of the gripper on the catheter by pulling or pushing sheath 42 to move the gripper respectively towards the proximal end 25 or distal end 24 of catheter 22. Removing pulling force F applied to control cable 41 relocks the gripper to catheter 22.

By way of a numerical example, assume that in the locked state clutch 60 is characterized by an inter-arm angle θ equal to 90°, a length L=4 Fr (French) (1.33 mm), a width W=L=4 Fr (1.33 mm), and that the clutch is formed from a fully hardened stainless steel wire having thickness τ=0.5 Fr (≈0.166 mm) A “French” is a unit measure of length equal to ⅓ of a mm (millimeter) that is conventionally used to express dimensions of catheters, features of catheters, and devices associated with the use of catheters. As with respect to the dimensions noted above in Fr, in the following discussion, a dimension given in Fr, is immediately followed by parentheses giving its equivalent value in mm

A minimum inner spread distance, “SDₘᵢn”, by which inside surfaces of distal arms 61 are separated, and by which proximal arms 62 are separated for the locked state of clutch 60 may be estimated by an expression SDₘᵢₙ=(W-2τ), which for the values of W and τ given above equals 3Fr (1 mm) If cowling 40 has an outer diameter (OD) equal to 6 Fr (2 mm), and is formed from stainless steel having thickness equal to about 0.15 Fr (0.05 mm), the cowling has an inner diameter (ID), I_{d}, equal to 5.7 Fr (1.90 mm) The inner diameter I_{d} of the cowling constrains how far proximal arms 61 can be spread apart and how far proximal arms 62 can be spread apart. Inner diameter I_{d} therefore constrains a maximum spread distance, SD_{Max}, between the inside surfaces of proximal arms 61 and between the inside surfaces of proximal arms 62 to a value estimated by an expression SD_{Max}=(I_{d}−2τ)=4.7 Fr (≈1.6 mm)

Assume clutch 60 releases a catheter when its spread distance, “SD” (distance between inside surfaces of distal arms 61 and between inside surfaces of proximal arms 62), is equal to a diameter “CatD” of the catheter plus a release distance “δ” equal 0.3 Fr (0.1 mm) Then clutch 60 is expected to be functional for gripping and releasing catheters characterized by diameters CatD that satisfy a constraint (SDₘᵢₙ+δ)≦CatD≦(SD_{Max}−δ), which expression for the above dimensions for clutch 60 requires 3.6 Fr (1.2 mm)≦CatD≦4.1 Fr (≈1.4 mm) Length and diameter of a gripper 30 that houses clutch 60 having the above dimensions are optionally equal to about 8F and 6 Fr respectively.

A gripper is of course not limited to the dimensions given in the above numerical example, and a gripper in accordance with an embodiment of the invention may have dimensions different from those in the example. For example, arterial and venous blood vessels in which catheter procedures may be performed have inner diameters that range from about 2 mm (6 Fr) for coronary arteries to about 60 mm (180 Fr) for the aorta. Interventional and/or diagnostic catheters used in the procedures may have outer diameters (ODs) from about 2.7 Fr, (≈0.9 mm), which may be used in coronary artery procedures, to about 24 Fr (8 mm), which may be used for procedures performed in the aorta. Relatively large interventional catheters having outer diameters between about 6 Fr (2 mm) and about 24 Fr (8 mm) are often used for procedures, such as trans-septal coronary ablation procedures, in the chambers of the heart. Grippers in accordance with embodiments of the invention adapted to grip catheters used in the procedures may have outer diameters in a range from less than or equal to 5 Fr (1.68 mm) to about 24 Fr (8 mm) and have components dimensioned to correspond to these diameters. In accordance with an embodiment of the invention a gripper has an outer diameter less than or equal to 6 mm Optionally, the diameter is less than or equal to 4 mm Optionally the diameter is less or equal to 2 mm.

Whereas gripper 30 is schematically shown comprising an X-shaped clutch 60, practice of embodiments of the invention are not limited to X-shaped clutches. FIGS. 2A and 2B schematically show topside and underside perspective views of a gripper 70 comprising a type of coil spring 72 whose coils are opposite facing “U-shaped” loops 74 formed having radii of curvature configured to grip a catheter, such as catheter 22. Coil spring 72 grips and locks gripper 70 to catheter 22 unless it is compressed by a pulling force applied to control cable 41 which proximally displaces cowling 40 relative to cowling mount 50. When compressed, loops 74 distort and their radii increase relaxing thereby their grip on catheter 22

FIG. 2C schematically shows a gripper 80 in which a clutch 82 comprises an inflatable “gripping” balloon 84 having a surface 86, and a control tube 88 for pumping a fluid into or out from the ripping balloon to respectively inflate and deflate the balloon. Gripper 80 is by way of example shown mounted to a stent delivery catheter 22. When inflated, as schematically shown in FIG. 2D, frictional forces between surface 86 of gripping balloon 84 and the surface of catheter 22 prevent gripper 80 from moving along the catheter and lock the gripper to the catheter. Deflating gripping balloon 84 of clutch 82 reduces frictional forces between the balloon and the catheter and unlocks gripper 80 from the catheter.

Various devices and instruments advantageous for use in procedures mediated by catheters may be attached to, or formed as a part of a gripper, in accordance with an embodiment of the invention. For example, a device or instrument may be bonded or otherwise coupled to cowling 40 or be integrally formed as a part of the cowling.

FIGS. 3A and 3B schematically show perspective views of a gripper 90, optionally similar to gripper 30, comprising an occlusion balloon 92 and an aspiration tube 94, in accordance with an embodiment of the invention. A fluid is pumped into or out from occlusion balloon 92 to respectively inflate or deflate the balloon via an inflation control tube 93. Gripper 90 and its balloon 92 and aspiration tube 94 are shown attached to a deployment catheter 22 of a stent deployment system 20 (SDS 20) comprising a stent 27 to conveniently add to the SDS a function of an embolism protection device (EPD). In FIG. 3A occlusion balloon 92 is deflated and in a condition in which gripper 90 and balloon 92 and aspiration tube 94 are pushed through a guiding catheter (not shown in the figure) to a site in a blood vessel of a patient at which they will be used in a procedure to deploy stent 27. In FIG. 3B the occlusion balloon is shown inflated, in a state after it has exited the guiding catheter and is in a position to block antegrade blood flow in the blood vessel and possible damage to the patient by particulate matter dislodged from the stenosis by deployment of the stent. FIGS. 5A-6C illustrate stent deployment procedures in which a gripper similar to gripper 90 is used to control blood vessel occlusion by particulate matter possibly dislodged by the procedures.

It is noted that occlusion balloon 92, which is shown enlarged in an inset 97, is formed to wrap around gripper 90 but leave a gap where lengthwise slot 34 is located so as not to interfere with clipping the gripper onto a stent. In accordance with an embodiment of the invention, ends of the balloon are formed having recessed pockets 95. One of the pockets, which is not normally seen in the perspective of FIG. 3A, is schematically shown in dashed lines. When the occlusion balloon is inflated the pockets “pop out” and meet each other along a boundary indicated by a line 96 in FIG. 3B, so that the inflated balloon completely surrounds gripper 90 and forms a substantially complete seal of a blood vessel in which it is positioned.

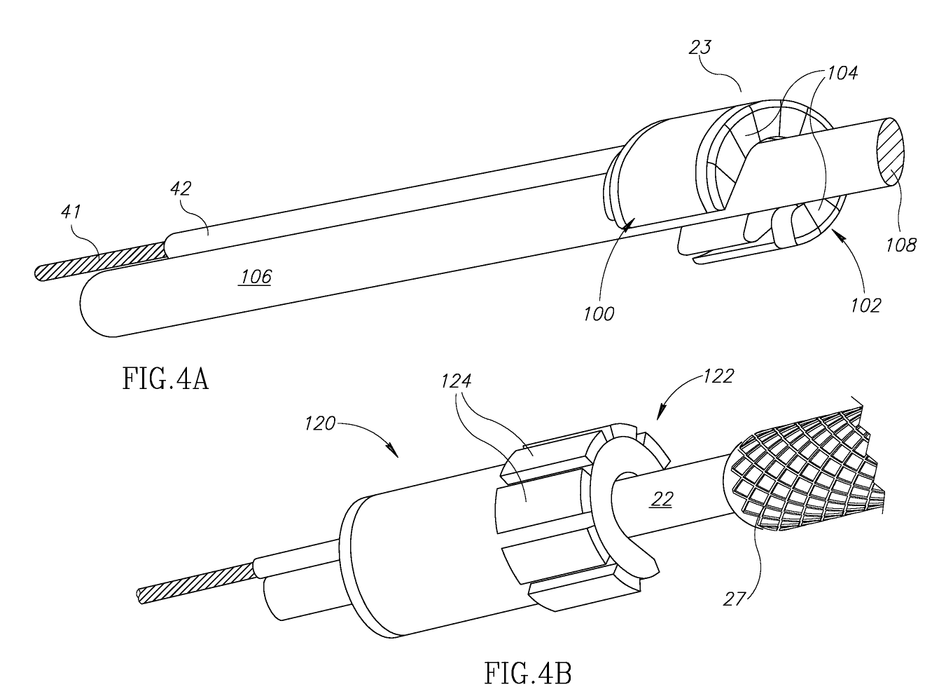

FIG. 4A schematically shows a gripper 100 comprising a planar, radial, phased array 102 of ultrasound transducers 104 mounted to a distal end 32 of the gripper in accordance with an embodiment of the invention. Gripper 100 is attached to a catheter 106 to acquire ultrasound images of a field of view (FOV) oriented forward of the catheter's distal end 23. Optionally, catheter 106 is used in a radio frequency (RF) ablation procedure to correct for atrial fibrillation and comprises an RF ablation transducer 108 at its distal end controllable to transmit RF energy sufficiently intense to ablate tissue. During the ablation procedure phased array 102 may be used to provide intracardiac echocardiography (ICE) images of the pulmonary veins, location of the atrial-venal junction, and location of RF transducer 108 relative to internal body features in its environment.

FIG. 4B schematically shows a gripper 120 comprising a cylindrical phased array 122 of ultrasound transducers 124, which is mounted by way of example to a catheter 22 of an SDS system 20 comprising a stent 27, in accordance with an embodiment of the invention. Phased array 122 is operable to provide intravascular ultrasound imaging (IVUS), for example to visualize positioning of stent 27 to treat an ostial stenosis.

FIGS. 5A-5I schematically illustrate an example of a scenario of a procedure for deploying a stent in a patient using an occlusion balloon and aspirator mounted to a gripper, in accordance with an embodiment of the invention.

FIG. 5A schematically shows a blood vessel 200 afflicted by a stenosis 202 located downstream of a junction 204 at which a “branch” blood vessel 206 branches off blood vessel 200. “Downstream” and “upstream” refer to a displacement or direction respectively in a direction of, and opposite to a direction of normal blood flow. The figure also shows an end 208 of a guiding catheter 210, which has been inserted into the patient by a member of a surgical team treating the patient, in preparation for deploying a stent in the stenosis. FIG. 5B schematically shows a guide wire 212 for use in guiding a stent deployment system (SDS) to stenosis 202 that has been pushed through the guide catheter and threaded through the stenosis.

FIG. 5C schematically shows a gripper 90 (FIGS. 3A and 3B) mounted and locked to a stent deployment catheter 22 comprising a collapsed stent 27 overlaying an expansion balloon 26 that has been guided by the surgical team through guiding catheter 210 over guide wire 212 to a location from which stent 27 can be positioned in stenosis 202. Gripper 90 comprises an occlusion balloon 92 connected to an inflation control tube 93, for temporarily blocking blood flow through blood vessel 200 to allow particulate debris that might be dislodged into the blood stream by deployment of stent 27 to be aspirated through an aspiration tube 94.

Prior to positioning stent 27 in stenosis 202 images of blood vessels 200 and 206 provided by a suitable imaging modality indicate that a distance D between stent 27 and occlusion balloon 92 is too large for effective use of the balloon and aspirator. For example, FIG. 5D schematically shows that for distance D, were stent 27 properly positioned in stenosis 202, occlusion balloon 92 would be located slightly upstream of junction 204. At the upstream location, were occlusion balloon 92 to be inflated, it might block blood flow not only to blood vessel 200, as desired, but also cause damage to the patient by unnecessarily blocking blood flow to branch blood vessel 206.

To adjust the location of occlusion balloon 92 relative to stent 27, gripper 90 is unlocked from catheter 22, optionally by pulling on a control wire 41 (FIGS. 1A-1G) connected to the gripper to unlock a clutch (for example, clutch 60, clutch 80) in the gripper from the catheter. After being unlocked, the gripper is moved to a position suitably closer to stent 27 by appropriately pushing, and if required also pulling, on sheath 42 that houses control wire 41 to move the gripper respectively towards or away from the stent. Releasing tension on control wire 41 then locks the repositioned gripper in place on the catheter and in condition to be deployed together with stent 27. FIG. 5E schematically shows gripper 90 suitably repositioned at a distance D-AD closer to stent 27 so that the occlusion balloon can be effectively operated when the stent is located in stenosis 202.

Following repositioning and locking of gripper 90 to deployment catheter 22 as shown in FIG. 5E, the deployment catheter is pushed to advance stent 27 into stenosis 202 and position it for deployment. FIG. 5F schematically shows a configuration of stent 27, gripper 90 and occlusion balloon 92 after the stent has been suitably located in the stenosis. Balloon 90 is then inflated as schematically shown in FIG. 5G so that it fills blood vessel 200 and blocks blood flow through the blood vessel.

Inflating balloon 26 expands and deploys stent 27 so that the stent presses material in stenosis 202 to the walls of blood vessel 200 and opens up the blood vessel to blood flow. Upon expanding stent 27 to open up stenosis 202 particulate matter and embolisms that may have been released and generated by deployment of the stent are aspirated out of blood in blood vessel 200 downstream of occlusion balloon 92 through aspiration tube 94. Block arrows 214 in FIG. 5H schematically represent aspiration of the particulate matter. The occlusion balloon is then deflated to enable its removal and removal of guide wire 212 and guide catheter 210 from the patient's body.

FIGS. 6A-6C schematically illustrate another scenario of a procedure for deploying stent 27 using gripper 90 and occlusion balloon 92 attached to deployment catheter 22 to open stenosis 202 in blood vessel 200, in accordance with an embodiment of the invention.

In preparing to deploy stent 27, gripper 90 and its occlusion balloon 92 are clipped and locked onto deployment catheter 22 relatively close to a proximal end 28 of stent 27. Optionally, distal end 32 of gripper 90 is located within a 5 mm of the proximal end of the stent. The deployment catheter is then pushed through guiding catheter 210 along guide wire 212 (FIGS. 5A and 5B) so that stent 27 is located close to stenosis 202.

FIG. 6A schematically shows stent 27 after it has been positioned close to stenosis 202.

After positioning as shown in FIG. 6A, occlusion balloon 92 is inflated to lock balloon 92 and gripper 90 to blood vessel 200, as schematically shown in FIG. 6B. Gripper 90 is then disengaged from catheter 22 optionally for example, by applying appropriate tensile force to control cable 41. Upon being disengaged from the gripper, the catheter is pushed to move stent 27 into place in stenosis 202, as schematically shown in FIG. 6C. Thereafter, expansion balloon 26 is inflated too expand stent 27 and open up stenosis 202 to blood flow and then expansion balloon 26 is deflated, and the volume of blood downstream of inflated occlusion balloon 92 is aspirated as shown in FIG. 5H. Following appropriate aspiration occlusion balloon 92 is deflated as shown in FIG. 5I to allow removal of deployment catheter and guiding catheter form blood vessel 210.

FIGS. 7A-7D schematically illustrate a scenario for using a gripper in accordance with an embodiment of the invention, to treat a stenosis of a renal artery located at an opening, referred to as an ostium, of the renal artery at a junction from which the renal artery branches off the abdominal artery.

FIG. 7A schematically shows a renal artery 300 of a kidney 301 having a stenosis 202 near an ostium 302 of the renal artery at a junction 304 from which the renal artery branches off the abdominal artery 306. The figure shows a guiding catheter 210 after it has been positioned in the abdominal artery and a guide wire 212 pushed through the catheter and threaded through the stenosis. FIG. 7B schematically shows a stent delivery system comprising a stent 27 mounted to a deployment catheter 22 to which a gripper 320 comprising an ostial positioner 322 has been attached in accordance with an embodiment of the invention. Gripper 320 may be any gripper controllable to be locked to, and unlocked from a catheter, in accordance with an embodiment of the invention. An ostial positioner is a device fixed to a deployment catheter at a desired distance from a distal end of the catheter that is configured to seat at an ostium of a blood vessel and allow the catheter to be pushed into the blood vessel only up to where the positioner is located on the catheter. The positioner thereby enables a stent mounted on the catheter to be positioned in the blood vessel accurately at a desired distance from the ostium.

By way of example, in FIG. 7B ostial positioner 322 comprises a balloon 323 and an inflation tube 324 through which a fluid can be pumped into or out from the balloon to respectively inflate and deflate the balloon. In FIG. 7B balloon 323 is shown deflated, just after deployment catheter 22 and gripper 320 have been pushed out along guide wire 212 from guiding catheter 210 and before the catheter is pushed along the guide wire into renal artery 300. In FIG. 7C ostial positioner balloon 323 is inflated to a diameter greater than a diameter of ostium 302 to prepare for pushing deployment catheter 22 and stent 27 into renal artery 300 and position the stent in stenosis 202. Gripper 320 is locked to deployment catheter 22 at a distance from stent 27 so that when deployment catheter 22 is pushed into renal artery 300 to a distance at which inflated ostial positioner balloon 323 seats in ostium 302 and prevents further ingress of the catheter into the artery, stent 27 is properly located in stenos 202. FIG. 7D schematically shows catheter 22 when the catheter has been pushed into the renal artery 300 to a distance at which ostial positioner balloon 323 seats in the ostium and stent 27 is properly located in stenosis 202. After positioning, the stent is expanded to open the stenosis, and thereafter ostial positioner balloon 323 is deflated and the deployment catheter and guiding catheter removed from the patient. Optionally, after positioning and expanding the stent 27 to open the stenosis and after the ostial positioner balloon 323 has been deflated, the gripper 320 may be unlocked from the catheter 22 and removed from the patient to allow the physician using the catheter 22 to continue using the catheter within the patient without the gripper 320. Such removal of the gripper 320 may enable the physician to further insert the catheter 22 for example into the renal artery 300 (or any other artery) to e.g. open or treat an additional stenosis that may be located downstream. Without removal of the gripper 320 and its positioner balloon 323 such operation may not be possible as the gripper 320 and its positioner balloon 323 may cause an interference that wouldn't enable such operation.

In the above descriptions of embodiments of the invention, a gripper that comprises a device to be attached to a catheter comprises in a same unit, the device and a clutch controllable to selectively lock the gripper to, and unlock the gripper from, a desired location along the catheter. In some embodiments of the invention gripper, hereinafter referred to as a “split gripper”, the function of attaching a device to a catheter and the function of locking the device in place are performed by different components. A “sliding collar” that is clipped onto a catheter attaches a device to a catheter and is introduced into a body together with a distal end of the catheter when the catheter is used to perform a procedure in the body. A separate proximal clutch remains outside the body when the catheter is used and is controllable to selectively lock and unlock the catheter at a desired location along the catheter.

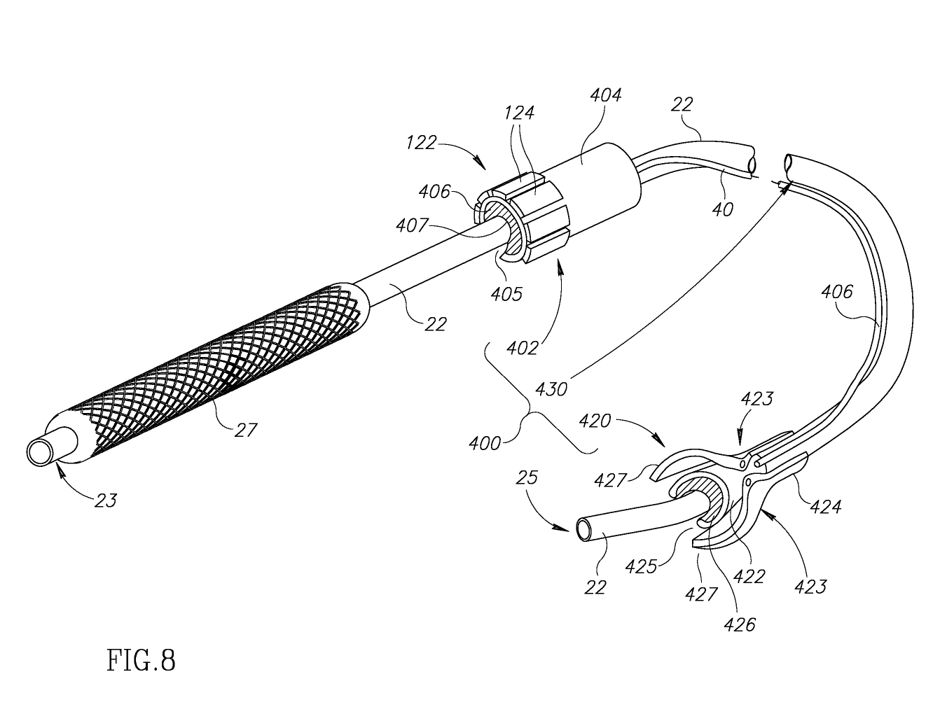

FIG. 8 schematically shows a split gripper 400 comprising a sliding collar 402, a proximal clutch 420, and a coupler 430 that connects the sliding collar to the proximal clutch, in accordance with an embodiment of the invention. By way of example, sliding collar 402 is mounted to a deployment catheter 22 comprising a stent 27 adjacent a distal end 23 of the catheter and comprises a phased array 122 of ultrasound transducers 124 for imaging internal features of a blood vessel into which the distal end of the catheter is introduced.

Sliding collar 402 comprises an optionally cylindrical shell 404 formed having a slot 405 and is filled with an elastic insert 406 formed having a recess 407, only and edge of which is shown in the figure, from a pliable, elastic material such as a spongy plastic or rubber. Sliding collar 402 is mounted to catheter 22 by pressing the collar to the catheter so that the catheter clips into the collar through slot 405 and seats into recess 407 in the insert. The insert is formed so that while it hold catheter 22 snugly in place in the sliding collar it allows the collar to slide along the catheter.

Proximal clutch 420 is mounted close to a proximal end 25 of catheter 22 and optionally comprises a support collar 422 supporting two opposing clamping arms 423 that are spring-loaded so that distal ends 424 of the arms are resiliently urged toward each other, or are elastically biased towards each other. Any of various methods and devices known in the art may be used to spring load or elastically bias clamping arms to support collar 422. For example, the arms may be loaded by a torsion spring or elastically biased by being formed integrally from an elastic plastic in a configuration for which the distal ends are tilted toward each other. Support collar 422 is optionally formed so that it may be mounted to catheter 22 similarly to the manner in which sliding collar 402 is mounted to the catheter—by pressing the clutch to the catheter so that the catheter clips into the clutch through a slot 425 to seat in an insert 426. Proximal clutch 420 locks by default onto catheter 22 upon being mounted to the catheter because spring loaded clamping arms 423 grip and hold the catheter. To unlock proximal clutch 420 proximal ends 427 of the arms are manually pressed towards each other.

Coupler 430 comprises a tube or wire that connects sliding collar 402 to clutch 420 and is sufficiently flexible to be threaded through a guiding catheter together with deployment catheter 22, but sufficiently stiff so that it doesn't buckle and bend away the catheter when they are inside the catheter. As a result, when proximal clutch 420 is unlocked from the catheter, if the clutch is moved along the catheter, sliding collar 402 moves with the clutch, and the clutch can be moved along the catheter to accurately position sliding collar 402 and phased array 122 at a desired location along the catheter. In FIG. 8 by way of example, coupler 430 comprises a tube 432 housing signal wires 433 for controlling phased array 122.

In the description and claims of the present application, each of the verbs, “comprise” “include” and “have”, and conjugates thereof, are used to indicate that the object or objects of the verb are not necessarily a complete listing of components, elements or parts of the subject or subjects of the verb.

Descriptions of embodiments of the invention in the present application are provided by way of example and are not intended to limit the scope of the invention. The described embodiments comprise different features, not all of which are required in all embodiments of the invention. Some embodiments utilize only some of the features or possible combinations of the features. Variations of embodiments of the invention that are described, and embodiments of the invention comprising different combinations of features noted in the described embodiments, will occur to persons of the art. The scope of the invention is limited only by the claims

1. A fastener for attaching a device to a catheter used to mediate a procedure in a patient's body, the fastener dimensioned so that it and the device can be introduced into the patient's body with the catheter and comprising:
a clutch controllable to lock the fastener onto and to unlock the fastener from the catheter; and
a controller operable to control the clutch selectively to lock onto and unlock from the catheter when the catheter and fastener are inside a body of a patient.

2. A fastener according to claim 1 wherein the controller is operable to reposition the clutch from a first location along the catheter when the clutch is unlocked and locate and lock the clutch and fastener at a new second location along the catheter.

3. A fastener according to claim 1 formed having a slot through which the catheter may be pressed to seat the catheter in the clutch.

4-6. (canceled)

7. A fastener according to claim 1, wherein the clutch comprises a resilient element having first and second states in one of which states the clutch is locked onto the catheter, and in the other of the states the clutch is unlocked from the catheter.

8. A fastener according to claim 7 wherein the resilient element comprises a compressive element which in a first state generates frictional forces that lock the fastener to the catheter and in the second state unlocks the fastener from the catheter.

9. A fastener according to claim 7 wherein the resilient element comprises a wire form having surface regions that press on the catheter in the first state to generate the frictional forces.

10-11. (canceled)

12. A fastener according to claim 9 wherein the wire form comprises two pairs of wire arms joined at a junction to form an X.

13-14. (canceled)

15. A fastener according to claim 9 wherein the wire form comprises opposite facing U-shaped loops curved to press on the catheter from opposite sides of the catheter to generate the frictional forces.

16. A fastener according to claim 15 wherein when compressed relative to the first state, a radius of curvature of the loops increase to reduce the frictional forces.

17. A fastener according to claim 7 and comprising a cowling that houses the clutch.

18. A fastener according to claim 17 and comprising a cowling mount on which the cowling is mounted so that it can slide back and forth along the cowling mount.

19. A fastener according to claim 18 wherein the resilient element is supported between the cowling and cowling mount.

20. A fastener according to claim 19 wherein the controller comprises a control cable housed in a sheath.

21. A fastener according to claim 20 wherein the sheath and control cable are connected to the cowling mount and cowling respectively, and tensile force applied to the cable that translates the control cable relative to the sheath in a direction away from the cowling unlocks the clutch.

22-23. (canceled)

24. A fastener according to claim 23 wherein the fastener has an outer diameter less than or equal to about 4 mm.

25. (canceled)

26. A fastener according to claim 25 wherein the fastener has an outer diameter equal to about 1.68 mm.

27. A fastener according to claim 1 any of the preceding claims and comprising an occlusion balloon and aspiration tube.

28-29. (canceled)

30. A fastener for attaching a device to a catheter used to mediate a procedure in a patient's body, the fastener dimensioned so that the fastener and device can be introduced into the patient's body with the catheter and comprising:
a collar attachable to the catheter so that it freely translatable along the catheter;
a clutch attachable to the catheter and controllable to selectively lock onto and be unlocked from the catheter; and
a connector that connects the collar and the clutch and maintains a substantially constant distance between them when both are attached to the catheter and the catheter is located in a guide catheter;
wherein the connector is sufficiently long so that the collar and clutch may be simultaneously located neat near distal and proximal ends of the catheter respectively.

31. (canceled)

32. A method of deploying a stent to treat a stenosis in a blood vessel, the method comprising:
introducing a deployment catheter comprising a stent, an aspiration tube, and an occlusion balloon locked to the catheter and located adjacent to the stent into the blood vessel;
inflating the occlusion balloon to block flow through the blood vessel and lock the balloon and catheter to the blood vessel;
while inflated, unlocking the occlusion balloon from the catheter;
moving the catheter to position the stent in the stenosis;
deploying the stent; and
aspirating blood in the blood vessel.
